(19) United States
(12) Patent Application Publication
(10) Pub. No.: US 2021/0277456 A1
(43) Pub. Date: Sep. 9, 2021

(54) METHOD FOR DETECTION OF MICROORGANISMS AND A FLUIDIC CHANNEL SYSTEM

(71) Applicant: Testo bioAnalytics GmbH, Titisee-Neustadt (DE)

(72) Inventors: Zinaida VASILEUSKAYA-SCHULZ, Freiburg (DE); Paul QUEHL, Gottingen (DE); Daniel BRAGA, Freiburg (DE); Joel RIEMER, Breitnau (DE); Serge RUDEN, Titisee-Neustadt (DE)

(73) Assignee: Testo bioAnalytics GmbH, Titisee-Neustadt (DE)

(21) Appl. No.: 17/174,672

(22) Filed: Feb. 12, 2021

(30) Foreign Application Priority Data
Feb. 14, 2020 (DE) 102020103957.3

(51) Int. Cl. C12Q 1/6841; G01N 1/30

(52) U.S. Cl. CPC C12Q 1/6841; G01N 1/30

(57) ABSTRACT

A method (7) for detecting microorganisms (1) in a sample (9), by delivery (10) of specific nucleic acid probes (11) into the individual microorganisms (1), which react (12) with the nucleic acid material present in the microorganisms (1), and subsequent optical detection (16) of the reaction products generated in the individual microorganisms (1), in which fixation (13) of the microorganisms (1) contained in the sample (9) is carried out before the delivery (10) of the nucleic acid probes (11) and a detergent (14) is added to the sample (9) before the fixation step (13) is completed.

### INCORPORATION BY REFERENCE

German Patent Application No. 10 2020 103 957.3, filed Feb. 14, 2020, is incorporated herein by reference as if fully set forth.

### TECHNICAL FIELD

The invention relates to a method for detecting microorganisms in a sample by means of delivery of specific nucleic acid probes into the individual microorganisms, which react with the nucleic acid material present in the microorganisms, and subsequent optical detection of the reaction products generated in the individual microorganisms, wherein fixation of the microorganisms contained in the sample is carried out at least during the delivery of the nucleic acid probes and a detergent is added to the sample before the fixation step is completed, and wherein the delivery and the fixation preferably take place concurrently.

### BACKGROUND

Known methods for the detection of nucleic acids in individual cells include, for example, in-situ hybridization (ISH). This involves using short synthetic nucleic acid probes which bind to the target sequence to be detected via base pairings. In-situ hybridization can be used for the specific detection of DNA and/or RNA molecules.

A variant of ISH technology in which the nucleic acid probes are fluorescently labeled is fluorescence in-situ hybridization (FISH). Carrying out conventional FISH methods for the specific detection of microorganisms with a particularly highly pronounced outer envelope comprises (can comprise) additionally a conditioning step prior to fixation and permeabilization. Conditioning destabilizes (can destabilize) the outer envelope of a cell to the extent that fixation and permeabilization agents can penetrate into the cell. For conditioning, microorganisms are (can be) typically treated with enzymes such as achromopeptidases, N-acetylmuramyl amidases, N-acetylmuramyl-alanine amidases, N—O-diacetylmuramyl amidases, glycylglycine endopeptidases, lyticases, chitinases, glucanases, cellulases, proteinases.

In the known FISH methods, the biological samples to be tested are thus first fixed and permeabilized to prepare for hybridization. Fixation “freezes” the current spatial structure of the microorganisms contained in the sample. In the case of permeabilization, the cell envelope of the microorganisms contained in the sample is made permeable to nucleic acid probes and to other externally added substances. The nucleic acid probes, which consist of an oligonucleotide and a label bound thereto, can then penetrate the cell envelope and bind to the target sequence in the cell interior. Fixation stabilizes macromolecules and cell structures and thus prevents the lysis of the cells during hybridization. At the same time, fixations permeabilize the cell envelope for the fluorescently labeled oligonucleotide probe molecules. Methanol, mixtures of alcohols, a low-percentage paraformaldehyde solution or a dilute formaldehyde solution are typically used for permeabilization/fixation. Despite these measures, the previously known methods cannot sufficiently establish the permeability of the membrane, and so the delivery of the detection probes into the microorganisms can be carried out only insufficiently. What may occur is overfixation of the cells. As a result of this, cell-envelope components are very highly crosslinked, and the cells are completely impermeable to nucleic acid probes. Moreover, the cell material may often be completely dissolved, for example if fixation was too short, and so the substances to be detected are detected in the dissolved state. This considerably hampers the detection of individual microorganisms. Another problem is that the aggregation of cells consistently occurs during fixation, said aggregation being dependent on bacterial strain and growth phase. This can hamper quantification of bacteria.

In order for the labeled nucleic acid probes to be able to hybridize to the nucleic acids in the cells, what is also additionally done is a denaturation step, so that the nucleic acids to be detected and the hybridization probes used are present in single-stranded form in the sample. Said denaturation can be done especially at high temperatures of about 90° C. to 100° C. However, to maintain the morphology of the samples, hybridization is typically carried out with use of formamide-containing solutions for denaturation of the double-stranded nucleic acids in the biological samples. In the previously known methods, the use of formamide lowers the melting temperature of double-stranded nucleic acids to 65° C. to 80° C. However, formamide-containing hybridization solutions are typically associated with very long reaction times.

After the fixation step, followed by a wash step in order to remove the interfering reagents, and permeabilization and denaturation steps, what occurs is the hybridization of the hybridization probes used to the nucleic acids contained in the sample, with the result that sequence-complementary segments can be found. The hybridization is then normally followed by a further wash step, and the fluorescence signals of the cells are subsequently evaluated under a fluorescence microscope. The evaluation can also be done cytometrically, for example by means of flow cytometry or solid-phase cytometry.

Due to the complexity of the conventional methods, which require a reagent exchange and multiple method steps, such methods are not suitable for a rapid and simple analysis. Moreover, due to toxicity, the use of formamide, paraformaldehyde and methanol is incompatible with nonlaboratory use.

### SUMMARY

Against this background, it is an object of the present invention to provide a simple and rapid method for detecting individual microorganisms in a sample, which method is performable in a cost-effective manner and outside laboratory environments.

The invention achieves this object through one or more features disclosed herein. In particular, what is therefore proposed according to the invention to achieve the stated object in a method of the kind described at the start is that a detergent is added to the sample to be tested before the fixation step is completed and that the delivery of specific nucleic acid probes and the fixation of the microorganisms preferably take place concurrently.

The advantage here is that, in the presence of a detergent, the permeability to the nucleic acid probes through the cell membranes is increased without the bacterial cell walls being destroyed at the same time. The morphological integrity of the cell is preserved, meaning that macromolecules such as DNA, RNA and ribosomes can remain in the cell. In the context of the present invention, “fixation” can be understood to mean a morphological fixation, i.e., a treatment in which a further alteration of the structure of the microorganisms is prevented. What is achievable here is immobilization of the inner structures of the microorganisms, preferably without crosslinking of the constituents, and so the microorganism is preserved as particles.

In relation to this, the invention takes advantage of the fact that effective penetration into the cell of nucleic acid probes labeled with fluorescent dyes or with relatively large enzyme molecules can be made possible by addition of a detergent to the sample.

In an advantageous embodiment according to the invention, the detergent is kept ready in a hybridization buffer. This can allow the addition of the detergent before the cells have been fixed, by the hybridization buffer already containing the two substances detergent and a fixation agent. As a result, the microorganisms can be permeabilized, i.e., rendered permeable for the delivery of the detection probes, at the same time as the fixation step or before the fixation step. As an alternative or in addition, the detergent can be kept ready in a preparation buffer.

Furthermore, in an advantageous embodiment according to the invention, the detergent is an ionic detergent, preferably cetyltrimethylammonium bromide, sodium deoxycholate or sodium dodecyl sulfate (SDS). The detergent can, for example, also be a nonionic detergent, preferably Triton X-100 or Tween 80. As an alternative or in addition, a zwitterionic detergent, preferably CHAPS, can also be used. In an advantageous embodiment, the concentration of the denaturing substance is 0-10 M, preferably 4.5-8 M, particularly preferably 5-6 M in the case of microorganisms without a conditioning step or 0 M in the case of microorganisms with a conditioning step.

In an advantageous embodiment according to the invention, the detergent is not added before the start of fixation. As an alternative or in addition, the detergent can be added before the start of fixation, for example with the sample or in the preparation buffer. What can be achievable by the addition of the detergent before the fixation step is completed is that nonlysed, individual microorganisms can be subsequently detected. As a result of the fixation of the microorganism, the cell boundary can be preserved in order to separate inside and outside at least until optical measurement and to prevent constituents of the microorganism from leaving it.

Furthermore, in an advantageous embodiment according to the invention, the hybridization buffer contains at least one denaturing substance. As an alternative or in addition, a buffer substance and/or a fixation agent can also be contained in the hybridization buffer. In particular, the fixation can be effected by the denaturing substance. This can allow an advantageous composition of the hybridization buffer, in which the same agent can be used for fixation and denaturation.

In an advantageous embodiment according to the invention, the denaturing substance contains a nontoxic substance. In particular, a nontoxic substance is guanidinium chloride and/or urea. Other substances, such as, for example, guanidinium thiocyanate and/or formamide, can also be used. However, the use of urea is preferred. This can ensure that the use of urea instead of the toxic formamide allows the application of the method according to the invention without a laboratory. The advantage here is that the reagents can be stored in dry form and subsequently disposed of as domestic waste. In an advantageous embodiment, the concentration of the denaturing substance is 4-10 M, preferably 4.5-8 M, particularly preferably 5-6 M.

In an advantageous embodiment according to the invention, the hybridization buffer contains tris(hydroxymethyl)aminomethane hydrochloride (TRIS-HCl) as buffer substance. The advantage here is that the buffer substance can stabilize the pH of the buffer between 6.5 and 9.0, preferably between 6.8 and 8.9, particularly preferably between 7.4 and 8.7. As an alternative or in addition, the buffer can be selected from veronal acetate buffer, HEPES buffer, PBS buffer, MES buffer, MOPS buffer, citrate buffer, barbital acetate buffer, TBS buffer, TE buffer, TAE buffer and TBE buffer. In an advantageous embodiment, the concentration of the buffer substance is 1-100 mM, preferably 2-50 mM, particularly preferably 5-20 mM.

Furthermore, in an advantageous embodiment according to the invention, the hybridization buffer contains at least one or more salts, preferably sodium chloride. The use of salt in the hybridization buffer can stabilize the double-stranded hybrids composed of probe and RNA. This means that the effect of denaturing substances can thereby be counteracted and the hybridization efficiency can thus also be increased. As an alternative or in addition, other salts such as, for example, magnesium chloride and/or potassium chloride can also be present. In an advantageous embodiment, the concentration of the salt is 700-1500 mM, preferably 750-1400 mM, particularly preferably 800-900 mM in the case of microorganisms without a conditioning step or 1200-1300 mM in the case of microorganisms with a conditioning step.

In addition, the hybridization buffer can contain at least one chelating agent, preferably ethylenediaminetetraacetic acid (EDTA). Protection from nucleases can therefore be ensured. As an alternative or in addition, the chelating agent can be selected from bisethylenediamine (salen), triethylenetetramine (TETA), triaminotriethylenetetramine (tren), ethylenediamine (en), ethylenediaminetriacetate (ted), diethylenetriaminepentaacetate (DTPA), triethylenetetraminehexaacetate (TTHA), oxalate (ox), tartrate (tart), citrate (cit). In an advantageous embodiment, the concentration of the chelating agent is 0-10 mM, preferably 0.1-5 mM, particularly preferably 0.5-1 mM.

In an advantageous embodiment according to the invention, the detection comprises a step of quantification of the microorganisms with hybridized nucleic acid probes. As an alternative or in addition, it is also possible to carry out single-detection of the microorganisms with hybridized nucleic acid probes. Absolute quantification of the organisms to be detected on the basis of particle measurement can therefore be made possible.

Furthermore, in an advantageous embodiment according to the invention, the nucleic acid probe is complementary to a DNA and/or RNA of a microorganism to be detected. Said nucleic acid probe can be selected from mono-labeled probes, dual-labeled probes, tetra-labeled probes, multi-labeled probes, molecular beacons and Scorpions probes. Preferably, the nucleic acid probes can be designed as quenched molecular beacons. What is achievable as a result is a higher fluorescence intensity and also a better signal-to-noise ratio, which may be advantageous especially for an automated application.

In fluorescence in-situ hybridization, an excessively high amount of fluorescently labeled hybridization probe can lead to increased background fluorescence. In an advantageous embodiment, the concentration of the nucleic acid probe is therefore 0.05-2 μM, preferably 0.1-1 μM, particularly preferably 0.13 μM.

Furthermore, in an advantageous embodiment according to the invention, the nucleic acid probe is connected to an optically detectable label. The detectable label can be selected especially from fluorescent labels, chemiluminescent labels, affinity labels and enzymatically active groups. Optical detection is therefore achievable. The affinity label can, for example, include biotin-streptavidin or antigen-antibody affinity binding pairs. The enzymatic label can, for example, be peroxidase, preferably horseradish peroxidase, or phosphatase, preferably alkaline phosphatase.

It is particularly advantageous when any background fluorescence or nonspecific fluorescence arising in the FISH methods described here is reduced or eliminated. As a result, automated detection methods in particular can operate more specifically or with a better detection limit. Nonspecific fluorescence can be brought about by various circumstances. These include:
The incomplete quenching efficiency of the quencher molecules used. Especially at high concentrations of FISH probes used, this gives rise to a high nonspecific background fluorescence due to FISH probes which are in excess (not bound to the target RNA/DNA).
The FISH probes can bind nonspecifically to non-target sequences or are incompletely closed and therefore not quenched or incompletely quenched.

As countermeasures for the abovementioned background fluorescence and for significant improvement of the signal-to-noise ratio, the following methods can therefore be applied (one method or a combination of these methods):
Fluorescence cascade: In accordance with/in analogy to Förster resonance energy transfer, a second dye is integrated into the method. The sample is irradiated with a light source of a wavelength through which the dye/reporter of the FISH probes does not fluoresce and therefore does not generate background fluorescence. Instead, a second dye (here: “fluorescence donor” with donor fluorophore) is introduced into the assay, which fluoresces owing to the introduced excitation wavelength and the emitted light (due to the fluorescence shift into a longer-wavelength range) is in turn capable of exciting the dye of the, for example, FISH probes/molecular beacons (here: “fluorescence acceptor” with acceptor fluorophore) to cause fluorescence. The emission spectrum in which the donor fluorophore fluoresces is beyond or only slightly within the detection spectrum/detection wavelengths of the detector and therefore does not generate relevant background fluorescence. The mechanism of this two-stage fluorescence only works or only works sufficiently when donor fluorophore and acceptor fluorophore are in sufficient spatial proximity and are not quenched by a quencher molecule. FISH probes used in this method can assume the fluorescence acceptor or the fluorescence donor function or else both functions. Ideally suitable here are combinations of oligos (DNA/RNA molecules) respectively provided with the donor fluorophore and acceptor fluorophore, which oligos are brought together in close proximity. In addition, binding sites within the target organism are chosen, which binding sites are adjacent or are close to one another owing to secondary structures and to which binding sites the oligos (fluorescence donor and fluorescence acceptor) bind. The acceptor fluorophore or donor fluorophore can, however, also be a, for example, profluorescent dye and be converted by enzymes within the target cells to form a fluorophore usable for detection (e.g., carboxyfluorescein) or be taken up in certain cell structures (such as RNA, DNA, proteins and lipids) (dyes such as, for example, SYBR Green, ethidium bromide, Coomassie) or accumulate within the target organism (e.g., tetramethylrhodamine methyl ester) and thus likewise be brought in sufficient spatial proximity of the donor fluorophore or acceptor fluorophore. What may be particularly advantageous are methods which bring either the donor fluorophore or the acceptor fluorophore having a nonspecific target (the target can, for example, be RNA or DNA) in sufficient proximity of whichever is the other fluorophore, since costs for the specific synthesis of, for example, specific FISH probes can thus be avoided. At the same time, the method can be thus used in a standardized manner for the improvement of other FISH assays and independently of the specific target sequences thereof. Especially the use of fluorophore-labeled “random” oligos, such as, for example, “random hexamers”, or other random oligo sequences is advantageous, since a mixture of all possible (for example) oligo-hexamer sequence options (this is the meaning of “random hexamers”) can bind to all single-stranded sequence options of nucleic acids. These “random” oligos can be labeled with one or more dyes at the 3′ end and/or at the 5′ end. By using a combination of fluorescence donors and acceptors, it is also possible to generally dispense with the use of quenchers in the FISH method described here and to thus create more cost-effective FISH probes (without quencher molecules) and with identical specificity, since specificity can be achieved by the required spatial proximity of the bound dyes (e.g., two specific oligos with dyes). For the use of nonspecific fluorescence donors or acceptors, it should be ensured that they are introduced only after the annealing step (“binding step”) with the specific reagent (specific FISH probe), since they may otherwise occupy the binding positions of the specific FISH probes and lead to false-negative results. If use is made of profluorescent dyes which must first be converted by enzymes of the stained cells for example or only accumulate in cells with a sufficiently intact cell membrane, this can additionally allow an inference concerning the vitality of the target organisms to be labeled.
Quenching probes after annealing step: Falsely bound or insufficiently closed FISH probes (“molecular beacons”) can be quenched once more or with better efficiency. To this end, the step to anneal the FISH probes is (can be) followed by introducing further oligos which are complementary to the FISH probes used. Said oligos (here: “quencher oligos”) (can) bear one or more quencher molecules at the ends thereof and (can) bind to the FISH probes. The complementary sequence is (can be) longer than the hairpin-forming neck sequence of the FISH probes and therefore leads, after the annealing (“binding”) thereof to the FISH probes, to a stable linearized (two-strand) structure. In said structure, the fluorophores of the FISH probes are (can be) present, then, in a quenched state due to the quencher of the “quencher oligos” and (can) generate only a low background fluorescence.
Immobilization of free FISH probes prior to measurement: Excess FISH probes can be removed from the sample mix. To this end, prior to measurement and after the step to anneal the FISH probes to the target molecules to be detected, the sample mixture is (can be) guided across a surface which binds (can bind) the excess (non-target-sequence-bound FISH probes) and removes them from the mix prior to a measurement of the sample. Alternatively, bodies (such as, for example, “beads”) to which the excess FISH probes bind can also be added to the sample. Thereafter, said bodies together with the excess FISH probes are separated from the reaction mix. In both cases, the surfaces or bodies are (can be) functionalized. Either they are coated/functionalized with oligos (e.g., DNA or RNA fragments) complementary to the FISH probes or they are coated/functionalized with other aids (e.g., antibodies directed against the fluorophores or quenchers of the FISH probes). What can be typically used for this purpose are biotinylated complementary oligos which are bound to a streptavidin-coated surface. It is likewise possible to couple the FISH probes used with possible binding aids (such as, for example, biotin or streptavidin) right from the start and to functionalize the surfaces/bodies used for immobilization with a complementary binding aid (e.g., FISH probes coupled with biotin and surfaces coated with streptavidin). The sample is/can be flushed (multiple times) across such a functionalized surface/body and then removed. A large portion of the previously nonbound FISH probes is (can be) immobilized on said surface/body and therefore the background fluorescence which arises from the excess FISH probes is (can be) removed from the method.
Destruction of fluorescence of excess FISH probes prior to measurement: The fluorophores of the excess FISH probes (which are not bound to the target RNA/DNA) can be altered physically, chemically or biologically such that they exhibit no fluorescence relevant to measurement. This can, for example, be achieved by the addition of reagents (e.g., enzymes) (e.g., P450 monooxygenases) which, for example, modify (e.g., hydroxylate) aromatic structures of the fluorophores and therefore alter or prevent the relevant fluorescence properties. Such reagents are (can be) added only after the step to anneal the FISH probes. The method is (can be) chosen such that the fluorophores of the FISH probes bound within the target organisms are not affected, since they are, for example, protected by the cell membrane of the target organism and the fluorescence-inhibiting reagent therefore cannot get into the proximity thereof or cannot interact therewith.
Reduction of the fluorescent background by the use of “free” quencher molecules in concentrations greater than 1 mM. The high concentration of free quencher molecules brings about (can bring about) a preferential quenching or reduction of the fluorescence of free/excess oligo probes (e.g., linear, molecular beacon, scorpions, etc.) outside the labeled microorganisms. Quenching of the intracellularly bound fluorescence probes due to free quencher molecules is (can be) lessened due to the fact that diffusion of the free quencher molecules into the cell and distribution within the cell is hampered or prevented by the cellular constituents. The selection of the quencher molecules depends (can depend) on the dye to be quenched. Suitable for the dyes FAM, Alexa488, Atto488 and the like are, for example, the isomers of methyl red (para-methyl red, meta-methyl red, o-methyl red; 4- or 3- or 2-{[4-(dimethylamino)phenyl]diazenyl}benzoic acid).
Immobilization of the target organisms: In a particularly advantageous configuration of the measurement system (e.g., a “lab-on-a-chip” system), the target organisms can be retained and/or concentrated on structures such as filters and especially track-etched membranes. It is therefore possible to separate the target organisms from the rest of the sample mix and to additionally clear excess FISH probes from the target organisms using flushing substances while labeled target organisms are retained. The FISH method described here can also be configured such that the described reaction steps, through the sequential addition of all the required reagents, are carried out directly on the filter structure (e.g., track-etched membrane). This can also reduce the reagents used in terms of the amount of substance thereof and therefore save production costs. To this end, it is necessary to flush the reagents individually onto the filter structure (e.g., by pneumatic and/or centrifugal transport). Particularly advantageous are configurations in which the target organisms can be first flushed onto a filter structure and then eluted (within the microfluidic system), for example by elution/flushing with a liquid against the direction of flushing/from the filter side facing away from the target organisms. In terms of their properties (such as autofluorescence), the filter structure should be designed such that direct reading of the measurement result on the filter structure can also be achieved.
Metering within the fluidic system: The FISH method described here makes it possible to counterbalance/buffer broad deviations of introduced samples volumes. To this end, it is necessary/advantageous to add certain reagents and especially the FISH probes in great excess in order to have a sufficiently high concentration of FISH probes even at unexpectedly high sample volumes. However, a great excess of the FISH probes is (can be) also distinguished by a high background fluorescence. Therefore, the goal should be that of being able to work with the minimum and exactly harmonized concentration of FISH probes. To this end, what is (can be) carried out on a fluidic platform is first a metering step which standardizes (“meters”) the starting sample volume in a loss-free manner and by, for example, centrifugal force. Owing to the now known starting volume, the FISH probes and the rest of the reagents can be set exactly in terms of their concentration and thus used within their performance optimum. To this end, the sample to be tested is first introduced, for example by centrifugal force, and then topped up with an excess of a buffer liquid up to an overflow channel. Unrequired buffer liquid is discharged via an overflow channel. The position of the overflow channel is chosen such that the liquid volume is known up to overflow of this “metering chamber”. The design of the “metering chamber” ensures that no sample material is lost.

The FISH method described here makes (can make) it possible to state the vitality of tested organisms on the basis of the rRNA concentration thereof. The goal is to achieve rapid and highly specific differentiation between “living” and “dead” microorganisms. In general, the FISH method described here is based on the degradation of rRNA of dead microorganisms and synthesis of rRNA of living microorganisms for differentiation of the vitality of the microorganisms. However, the differing membrane permeability of living and dead organisms can likewise be utilized for differentiating living and dead organisms from one another relatively rapidly. The assumption here is that dead cells exhibit a distinctly increased permeability of the cell membrane. With the following options, it is possible to lower the detection threshold for “living” microorganisms, since dead microorganisms are no longer (sufficiently) labeled and living microorganisms (for their signal amplification or increasing of the difference between living and dead microorganisms) do not need to synthesize additional nucleic acids (e.g., rRNA) for living/dead differentiation. The method can therefore be significantly quickened:
Target depletion: The target nucleic acids (such as rRNA) can be degraded in dead microorganisms prior to the actual method. It is therefore possible to lower the detection threshold for “living” microorganisms and to wait for less rRNA synthesis (time) thereof, since dead microorganisms, owing to their missing target sequences, can no longer or hardly be labeled by the method. To this end, what can be added are either ribonucleases (such as ribonuclease A) alone or combinations of ribonucleases (e.g., ribonuclease H) with nucleic acids (e.g.: DNA oligos). In the first case, ribonuclease A degrades the total RNA accessible thereto. In the second case, the introduced nucleic acids specifically bind to the nucleic acids to be degraded (e.g., rRNA) and the ribonuclease H recognizes the heteroduplex of introduced DNA and target rRNA. Said heteroduplex is, then, specifically degraded by the ribonuclease H. In the case of the ribonucleases and nucleic acids, it is not possible to penetrate the membranes of living microorganisms—therefore, only nucleic acids of dead organisms having sufficiently permeable cell membranes are degraded. In addition, it is also possible to synthesize further structures (“anchor structures”) onto, for example, the DNA oligos, which further hamper the penetration of intact cell membranes. In general, it should be ensured in this approach that, prior to addition of the FISH probes, the introduced ribonucleases are inactivated, inhibited or removed from the sample mix (e.g., by flushing steps on a filter/track-etched membrane). In addition, this method can also be combined with detection methods which, for example, are based on Förster resonance energy transfer or allow the reading of multiple labels in one microorganism. For example, proteins or DNA can be used as fluorescence donor (or fluorescence acceptor) or as second target structure for labels and be degraded in dead cells before the start of the actual method (e.g., by proteinases or deoxyribonuclease, which cannot penetrate intact membranes of living organisms). Therefore, only living microorganisms are captured by the method, since either dead cells have no fluorescence donor (or fluorescence acceptor) and thus no, for example, fluorescence in the relevant emission range, or they have only the fluorescence of the FISH probes, but not the fluorescence of the living/dead indicator (e.g., DNA or proteins which are no longer present), and are thus only mono-labeled.
Target blocking: The differing membrane permeability of living and dead microorganisms can be utilized for introducing nucleic acid (e.g., DNA oligos) into dead microorganisms and for occupying the test-relevant binding sites thereof (e.g., in the rRNA thereof) therewith. Thereafter, FISH probes, for example, can no longer bind to these positions, since the sites are already occupied. It should be ensured that the oligos introduced for occupying the relevant binding sites are removed from the sample mix before introduction of the FISH probes and before permeabilization of the living microorganisms. This can, for example, be achieved by flushing the sample mix through a filter (e.g., track-etched membrane).

Use of living/dead stains: It is possible for the FISH method used here to be carried out with additional living/dead differentiation of the relevant microorganisms. To this end, the target organisms can be fixed on a filter (such as a track-etched membrane) and treated with a living/dead dye (such as, for example, propidium iodide). The membrane is mapped by a sensor and the state “living” or “dead” is recorded for the respective microorganisms. Afterwards or at the same time, the FISH method is carried out and the microorganisms positively labeled by the FISH probes are additionally provided with the state “living” or “dead” in the data acquisition. Furthermore, the microorganisms can also be labeled using the FISH method described here and additionally provided with a living or dead dye (e.g., propidium iodide) if it has spectral properties different from the dyes of the FISH probes. Thereafter, multiple spectral properties (e.g., fluorescences in different wavelengths or spectra) are (can be) read per target organism and information about, for example, organism species and the vitality thereof is (can be) recorded at the same time.

In an advantageous embodiment according to the invention, the method can be performed with a fluidic channel system, especially with a disk-shaped sample carrier. The advantage here is that specific detection of microorganisms can be made possible in different fields of application. For example, the method according to the invention can be used for microbiological food control, hygiene control, clinical and biotechnological applications and also environmental analysis.

A preferred application provides a fluidic channel system comprising means for carrying out the method, especially as described above and/or as per any of the claims directed to a method. For example, a detection zone and a preparation zone can be formed in the fluidic channel system for carrying out the method according to the invention. In particular, the cross-sections of the channels of the fluidic channel system can be matched to dimensions of the microorganisms.

The fluidic channel system can, for example, be designed as a sample carrier. The sample carrier according to the invention comprises especially at least one cavity containing a nucleic acid probe and at least one detergent. As an alternative or in addition, the sample carrier can comprise means for optical counting of labeled microorganisms.

The sample carrier according to the invention can be designed as a disk-shaped sample carrier. For example, the sample carrier can be designed as a planar sample carrier. The advantage here is that the disk shape of the sample carrier can utilize centrifugal force for fluid conveyance. Fluid conveyance is also achievable by means of pressure or in another way. As an alternative, the sample carrier can have a three-dimensional extent, for example in the form of a cylinder or in the style of a cuvette.

For example, the disk-shaped nature can have rotational symmetry. This can be advantageous for centrifugation. It is also alternatively possible to form rectangular sample carriers, as in the case of a chip card, or segment-shaped sample carriers, as in the case of a pizza slice.

### BRIEF DESCRIPTION OF THE DRAWINGS

The invention will now be described in more detail with reference to exemplary embodiments, without however being limited to said exemplary embodiments. Further exemplary embodiments arise from combination of the features of individual or multiple claims with one another and/or with individual or multiple features of the exemplary embodiments.

In the figures:

FIG. 1 shows a schematic depiction of a conventional method based on fluorescence in-situ hybridization (FISH) for microorganisms without a highly pronounced outer envelope,

FIG. 2 shows a schematic depiction of a FISH method according to the invention for microorganisms without a highly pronounced outer envelope,

FIG. 3 shows a detailed depiction of the method according to FIG. 2 for microorganisms without a highly pronounced outer envelope,

FIG. 4 shows a schematic depiction of a conventional method based on fluorescence in-situ hybridization (FISH) for microorganisms with a highly pronounced outer envelope,

FIG. 5 shows a schematic depiction of a FISH method according to the invention for microorganisms with a highly pronounced outer envelope, and

FIG. 6 shows a detailed depiction of the method according to FIG. 5 for microorganisms with a highly pronounced outer envelope.

### DETAILED DESCRIPTION

FIGS. 1 to 3 show different embodiments of a method for detecting microorganisms without a highly pronounced outer envelope.

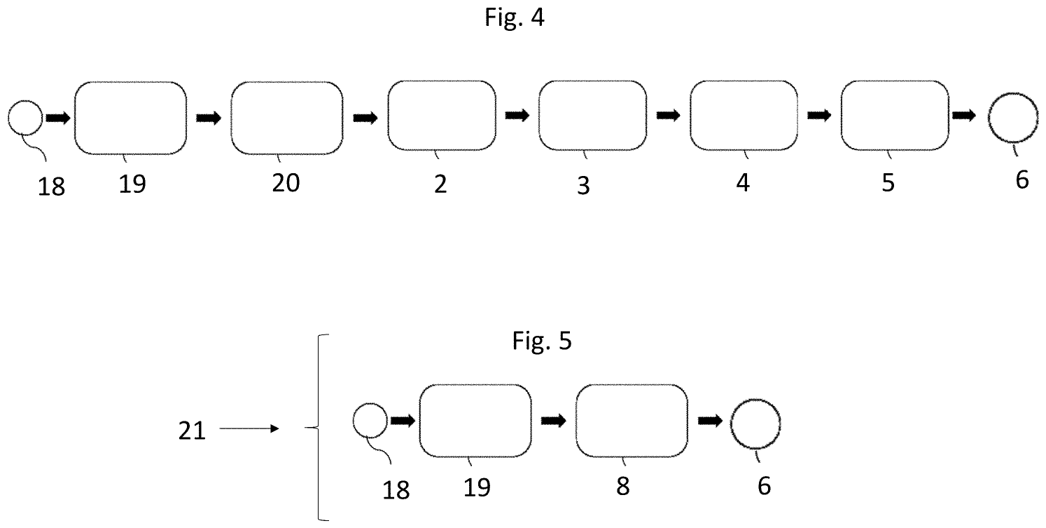

FIGS. 4 to 6 show different embodiments of a method for detecting microorganisms with a highly pronounced outer envelope.

FIG. 1 shows a conventional FISH method for specifically detecting nucleic acids in individual microorganisms 1, comprising the following steps: fixation and permeabilization 2 of the microorganisms 1 contained in a sample; washing 3 in order to remove the reagents used for permeabilization; incubation of the fixed and permeabilized microorganisms 1 with nucleic acid probes in order to bring about hybridization 4; removal or wash-away 5 of the nonhybridized nucleic acid probes; and subsequent analysis 6 of the microorganisms 1 hybridized to the nucleic acid probes.

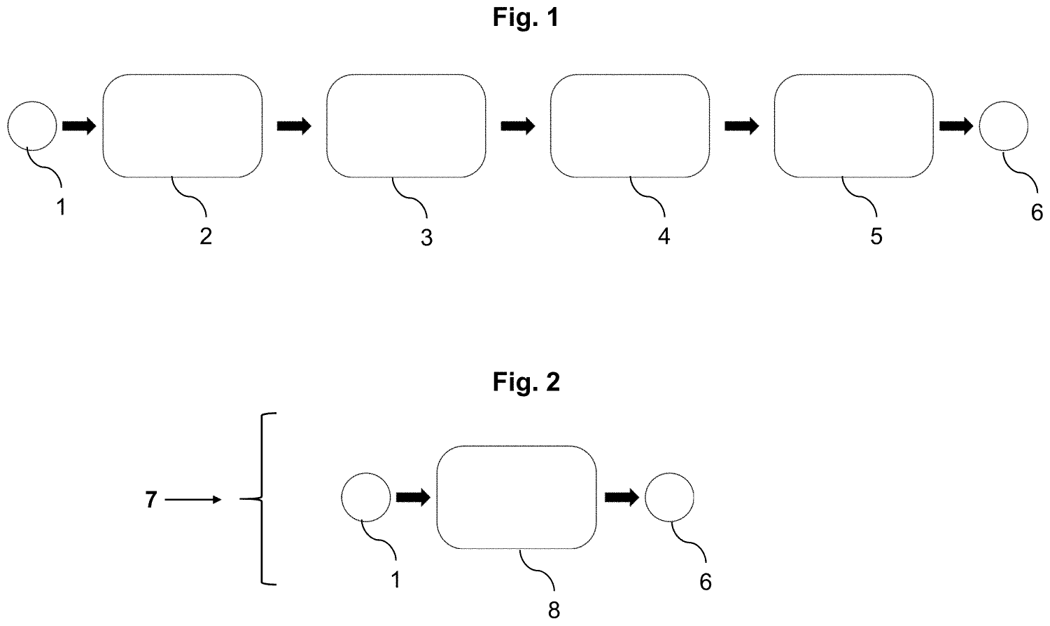

FIG. 2 shows a method 7 according to the invention for specifically detecting microorganisms 1, combining the steps of permeabilization, fixation and hybridization into a single method step 8. Additional wash steps or buffer exchanges are not required.

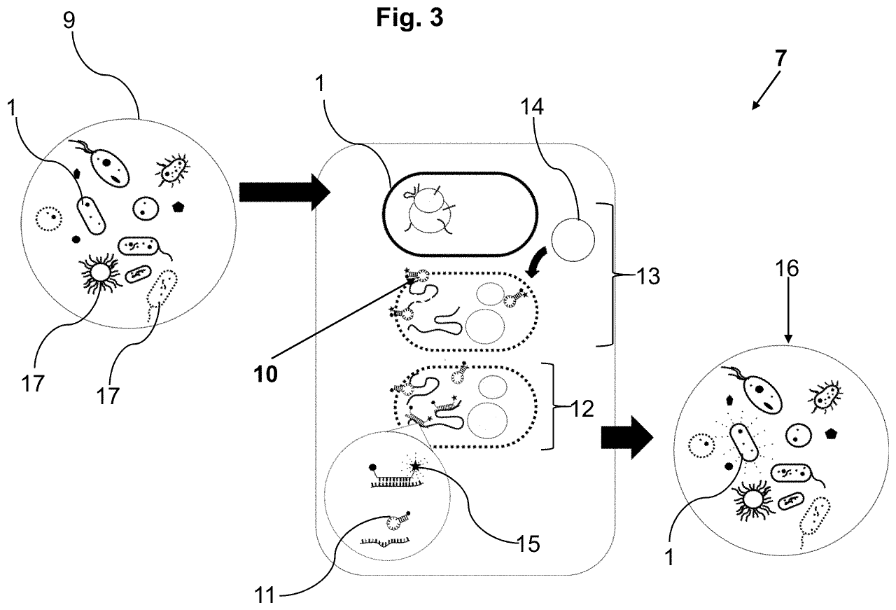

FIG. 3 shows a method 7 according to the invention for detecting microorganisms 1 in a sample 9 by means of delivery 10 of specific nucleic acid probes 11 into the individual microorganisms 1, the nucleic acid probes 11 reacting with the nucleic acid material present in the microorganisms 1. What therefore takes place is hybridization 12.

It is additionally evident from FIG. 3 that fixation 13 of the microorganisms 1 contained in the sample 9 is carried out at least during the delivery 10 of the nucleic acid probes 11, thereby preventing further alteration of the structure of the microorganisms. It is preferred that the delivery 10 and the fixation 13 take place concurrently.

To allow effective penetration of nucleic acid probes 11 into the cells without the bacterial cell walls being destroyed at the same time and the morphological integrity of the cells thus being preserved, a detergent 14 is added to the sample 9 before the fixation step 13 is completed (cf. FIG. 3). Absolute quantification of the organisms to be detected on the basis of particle measurement is therefore achievable.

As is additionally apparent from FIG. 3, the nucleic acid probe 11 is connected to an optically detectable label 15 in order to allow subsequently the optical detection 16 of the reaction products generated in the individual microorganisms 1.

In a preferred application, specific detection of microorganisms without a highly pronounced outer envelope is achieved by admixing a microorganism-containing sample with a hybridization buffer (900 mM NaCl, 20 mM Tris/HCl, 0.01% SDS, 5.3 M urea, 1 mM EDTA, 0.13 μM hybridization probe and pH 8.0) and incubating it at a temperature of 52° C. for a period of from 15 to 90 minutes. Following the end of this incubation time, the samples in which hybridization is completed are analyzed by cytometry or fluorescence microscopy.

According to the invention, what is therefore proposed is to provide a method 7 for detecting microorganisms 1 in a sample 9, by means of delivery 10 of specific nucleic acid probes 11 into the individual microorganisms 1, which react 12 with the nucleic acid material present in the microorganisms 1, and subsequent optical detection 16 of the reaction products generated in the individual microorganisms 1, wherein fixation 13 of the microorganisms 1 contained in the sample 9 is carried out before the delivery 10 of the nucleic acid probes 11 and a detergent 14 is added to the sample 9 before the fixation step 13 is completed.

FIG. 4 shows a conventional FISH method for specifically detecting nucleic acids in individual microorganisms with a highly pronounced outer envelope 18, comprising the following steps: enzymatic conditioning 19 of the microorganisms 18 contained in a sample; washing 20 in order to remove the reagents used for conditioning; fixation and permeabilization 2 of the microorganisms 18 contained in a sample; washing 3 in order to remove the regents used for permeabilization; incubation of the conditioned, fixed and permeabilized microorganisms 18 with nucleic acid probes in order to bring about hybridization; removal or wash-away 5 of the nonhybridized nucleic acid probes; and subsequent analysis 6 of the microorganisms 18 hybridized to the nucleic acid probes.

FIG. 5 shows a method 21 according to the invention for specifically detecting microorganisms with a highly pronounced outer envelope 18, containing the steps of conditioning 19, permeabilization, fixation and hybridization, but combining the steps of permeabilization, fixation and hybridization into a single method step 8. Additional wash steps or buffer exchanges are not required.

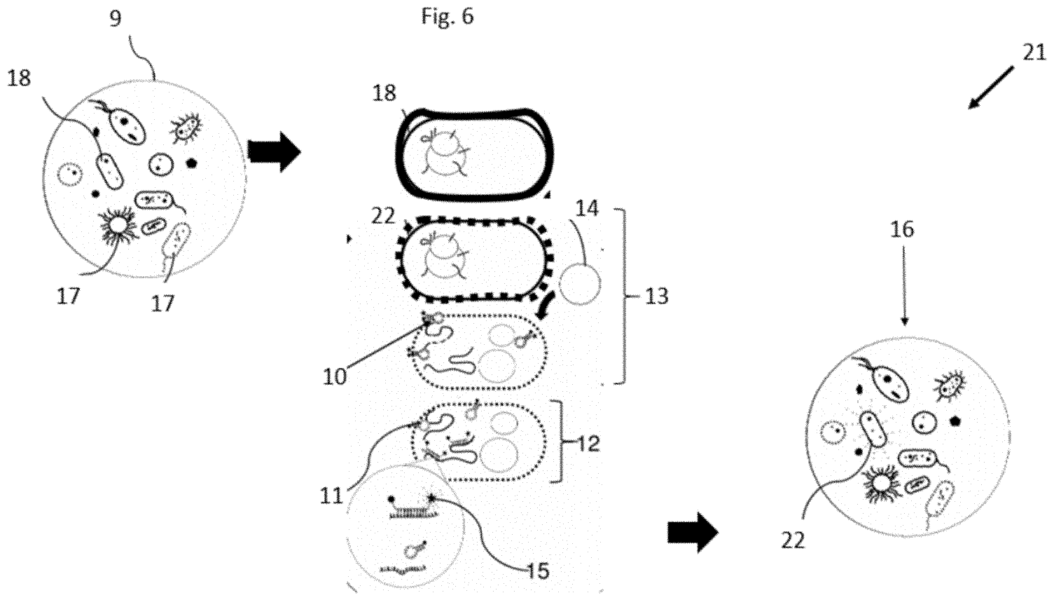

FIG. 6 shows a method 21 according to the invention for detecting microorganisms with a highly pronounced outer envelope 18 in a sample 9 by means of delivery 10 of specific nucleic acid probes 11 into the individual conditioned microorganisms 22, the nucleic acid probes 11 reacting with the nucleic acid material present in the conditioned microorganisms 22. What therefore takes place is hybridization 12.

It is additionally evident from FIG. 6 that fixation 13 of the microorganisms 22 which are contained in the sample 9 and conditioned is carried out at least during the delivery 10 of the nucleic acid probes 11, thereby preventing further alteration of the structure of the microorganisms. It is preferred that the delivery 10 and the fixation 13 take place concurrently.

To allow effective penetration of nucleic acid probes 11 into the cells without the bacterial cell walls being completely destroyed at the same time and the integrity of the cells thus being preserved, a detergent 14 is added to the sample 9 after the conditioning before the fixation step 13 is completed (cf. FIG. 6). Absolute quantification of the organisms to be detected on the basis of particle measurement is therefore achievable.

As is additionally apparent from FIG. 6, the nucleic acid probe 11 is connected to an optically detectable label 15 in order to allow subsequently the optical detection 16 of the reaction products generated in the individual conditioned microorganisms 22.

In a preferred application, specific detection of microorganisms with a highly pronounced outer envelope is achieved by incubating a microorganism-containing sample with one enzyme and/or multiple enzymes for the purpose of conditioning at a temperature of 52° C. for from 10 to 30 minutes. This is directly followed by admixing the sample with a hybridization buffer (1250 mM NaCl, 20 mM Tris/HCl, 0.01% SDS, 1 mM EDTA, 0.13 μM hybridization probe and pH 8.0) and incubating it at a temperature of 52° C. for a period of from 20 to 90 minutes. Following the end of this incubation time, the samples in which hybridization is completed are analyzed by cytometry or fluorescence microscopy.

According to the invention, what is therefore proposed is to provide a method 21 for detecting microorganisms with a highly pronounced outer envelope 18 in a sample 9, by means of delivery 10 of specific nucleic acid probes 11 into the individual preconditioned microorganisms 22, which react 12 with the nucleic acid material present in the microorganisms 22, and subsequent optical detection 16 of the reaction products generated in the individual microorganisms 22, wherein fixation 13 of the microorganisms 18 contained in the sample 9 is carried out before the delivery 10 of the nucleic acid probes 11 and a detergent 14 is added to the sample 9 before the fixation step 13 is completed.

### LIST OF REFERENCE SIGNS

1 Microorganisms to be detected without a highly pronounced outer envelope
2 Fixation and permeabilization according to a conventional FISH method
3 Washing according to a conventional FISH method
4 Hybridization according to a conventional FISH method
5 Washing according to a conventional FISH method
6 Analysis
7 Method according to the invention for microorganisms without a highly pronounced outer envelope
8 Permeabilization, fixation and hybridization, comprised in one method step
9 Sample containing microorganisms
10 Delivery of specific nucleic acid probes
11 Nucleic acid probe
12 Hybridization
13 Fixation of the microorganisms
14 Detergent
15 Label
16 Optical detection for microorganisms without a highly pronounced outer envelope
17 Further microorganisms which are not to be detected
18 Microorganisms to be detected with a highly pronounced outer envelope
19 Conditioning according to a conventional FISH method
20 Washing according to a conventional FISH method
21 Method according to the invention for microorganisms with a highly pronounced outer envelope
22 Microorganisms to be detected with a highly pronounced outer envelope after conditioning

1. A method (7) for detecting microorganisms without a highly pronounced outer envelope (1) in a sample (9), the method comprising:
delivering (10) specific nucleic acid probes (11) into the individual microorganisms (1), which react (12) with the nucleic acid material present in the microorganisms (1),
subsequently optically detecting (16) reaction products generated in the individual microorganisms (1),
carrying out a fixation (13) of the microorganisms (1) contained in the sample (9) at least during the delivery (10) of the nucleic acid probes (11), and
adding a detergent (14) to the sample (9) before the fixation step (13) is completed.

2. A method (21) for detecting microorganisms with a highly pronounced outer envelope (18) in a sample (9), the method comprising:
delivering (10) specific nucleic acid probes (11) into individual conditioned microorganisms (22), which react (12) with the nucleic acid material present in the individual conditioned microorganisms (22),
subsequently optically detecting (16) reaction products generated in the individual conditioned microorganisms (22),
carrying out a fixation (13) of the microorganisms (18) contained in the sample (9) at least during the delivery (10) of the nucleic acid probes (11), and
adding a detergent (14) to the sample (9) before the fixation step (13) is completed.

3. The method (7) as claimed in claim 1, wherein the detergent (14) is kept ready in at least one of a hybridization buffer or a preparation buffer.

4. The method (7) as claimed in claim 1, wherein the detergent (14) is at least one of an ionic detergent, a nonionic detergent preferably Triton X-100 or Tween 80, or a zwitterionic detergent.

5. The method (7) as claimed in claim 1, wherein the detergent (14) is at least one of not added before the start of fixation (13) or added before the start of fixation (13).

6. The method (7) as claimed in claim 3, wherein the hybridization buffer contains at least one of a denaturing substance or a buffer substance.

7. The method (7) as claimed in claim 6, wherein the fixation (13) is effected by the denaturing substance.

8. The method (7) as claimed in claim 7, wherein the denaturing substance contains a nontoxic substance.

9. The method (7) as claimed in claim 3, wherein the hybridization buffer contains at least one or more salts.

10. The method (7) as claimed in claim 1, wherein the detecting (16) comprises at least one of a step of quantification or of single-detection of the microorganisms with hybridized nucleic acid probes.

11. The method (7) as claimed in claim 1, wherein the nucleic acid probe (11) is complementary to at least one of a DNA or RNA of a microorganism (1) to be detected.

12. The method (7) as claimed in claim 1, wherein the nucleic acid probe (11) is selected from the group consisting of mono-labeled probes, dual-labeled probes, tetra-labeled probes, multi-labeled probes, molecular beacons and Scorpions probes.

13. The method (7) as claimed in claim 1, wherein the nucleic acid probe (11) is connected to an optically detectable label (15).

14. The method (7) as claimed in claim 1, wherein the method (7) is performed using a fluidic channel system.

15. The method (7) as claimed in claim 1, further comprising at least one of: a step for additional background reduction, the introduction of quenching oligos after the step to anneal the nucleic acid probes which are FISH probes, immobilization of free FISH probes prior to measurement, physical, chemical or biological alteration of fluorophores of excess FISH probes, use of “free” quencher molecules in concentrations greater than 1 mM, immobilization of the target organisms on structures, or addition of certain reagents in great excess (metering).

16. The method (7) as claimed in claim 1, further comprising at least one of: an additional initial step for improved living/dead differentiation by degradation of the target nucleic acids (such as rRNA) (target depletion) in dead microorganisms, introduction of nucleic acids (e.g., DNA oligos) into dead microorganisms in order to occupy test-relevant binding sites (e.g., in the rRNA thereof) therewith (target blocking), or use of living/dead stains.

17. A fluidic channel system for carrying out the method of claim 1, comprising the fluidic channel system comprising at least one of a cavity containing a nucleic acid probe (11) and at least one detergent (14), an optical counter for at least one of labeled microorganisms without a highly pronounced outer envelope (1) or labeled microorganisms with a highly pronounced outer envelope (18).

18. The method according to claim 1, wherein the delivery (10) and the fixation (13) take place concurrently.

19. The method according to claim 2, wherein the delivery (10) and the fixation (13) take place concurrently.

20. The method of claim 1, wherein the nucleic acid probe (11) comprises quenched molecular beacons.

21. The method of claim 13, wherein the detectable label (15) is selected from the group consisting of fluorescent labels, chemiluminescent labels, affinity labels and enzymatically active groups.
